(19) United States
(12) Patent Application Publication
(10) Pub. No.: US 2010/0021494 A1
(43) Pub. Date: Jan. 28, 2010

(54) HEPATITIS-RELATED PREVENTIVE OR THERAPEUTIC AGENTS

(76) Inventor: Isao Yuasa, Osaka (JP)

Correspondence Address: FOLEY & LARDNER LLP, 150 EAST GILMAN STREET, P.O. BOX 1497, MADISON, WI, 53701-1497, US

(21) Appl. No.: 12/366,966

(22) Filed: Feb. 6, 2009

(30) Foreign Application Priority Data
Jul. 25, 2008 (JP) 2008-192579

(51) Int. Cl. A61K 36/03

(52) U.S. Cl. 424/195.17

(57) ABSTRACT

The present disclosure relates to provide a novel hepatitis-related therapeutic or preventive agent. The present disclosure provides a hepatitis-related preventive or therapeutic agent including a phlorotannin.

### CROSS-REFERENCE TO RELATED APPLICATION

This application claims priority to Japanese Patent Application No. 2008-192579 filed on Jul. 25, 2008. The entire contents of which are hereby incorporated by reference in its entirety.

### TECHNICAL FIELD

The present disclosure relates to a novel hepatitis preventive agent and/or a hepatitis therapeutic agent.

### BACKGROUND

The main cause of cirrhosis has conventionally been thought to be persistent infection by hepatitis virus. However, cirrhosis can also result from alcoholic hepatitis.

In recent years, there has been a growing trend toward increased alcohol consumption by Japanese as well as an increase in the size of the alcohol-consuming population in Japan. This increased prevalence of alcohol consumption has led to a rapid increase in the incidence of alcoholic hepatitis, which has now become a serious social issue.

Alcoholic hepatitis is a disease characterized by deterioration and necrosis of liver cells associated with the consumption of alcohol. Alcoholic hepatitis is known to progress to alcoholic cirrhosis and liver cancer.

Phlorotannin has antioxidative action, vasodilatory action, and anti-inflammatory action as well as having preventative effects on heart disease (see, for example, Japanese Patent Application No. 2006-328010 and Japanese Patent Application No. 2002-212095). Phlorotannin also reduces the accumulation of fat in normal liver cells (See Emil Chi, “The Beneficial Effects of Feeding SEANOL-Based Drink “X2” in a Fat Mouse Model Study”).

### SUMMARY

The disclosure provides, for example, a novel hepatitis preventive agent and/or hepatitis therapeutic agent, and methods for treating and/or preventing hepatitis. As described herein, phlorotannin is a novel hepatitis preventive agent and/or hepatitis therapeutic agent, and is effective in treating and preventing hepatitis.

In one aspect, a hepatitis preventive agent or hepatitis therapeutic agent includes a phlorotannin. In one aspect, the method for preventing or treating hepatitis includes administering a phlorotannin. In the present disclosure, illustrative examples of the phlorotannin include, but are not limited to, phlorotannin extracted from brown algae and phlorotannin extracted from ecklonia cava and the like. In some embodiments, hepatitis includes alcoholic hepatitis. In some embodiments, hepatitis (optionally alcoholic hepatitis) is associated with cirrhosis and/or hepatic fibrosis. In some embodiments, hepatitis and/or fibrosis is associated with liver cancer.

In one aspect, the disclosure provides a liver cancer preventive agent and/or liver cancer therapeutic agent. In one aspect, the disclosure provides a method for preventing or treating liver cancer. In some embodiments, the liver cancer includes cancer caused by hepatitis.

The present disclosure provides a medicament, a food, and/or a beverage including one or more of the hepatitis preventive agent, the hepatitis therapeutic agent, the liver cancer preventive agent, and/or the liver cancer therapeutic agent. In some embodiments, the beverage is an alcoholic beverage including one or more of the hepatitis preventive agent, the hepatitis therapeutic agent, the liver cancer preventive agent, and/or the liver cancer therapeutic agent.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

FIG. 1 shows a graphical representation of the viability of liver cells in the alcoholic hepatitis model described in Example 1. The viability is shown as the mean±standard deviation thereof. The vertical axis indicates the cell viability of liver cells based on a value of 100 for a control group. The respective bars indicate, in order from the left, a control group (no ethanol added), an ethanol addition (100 mM) group, various concentrations of green tea polyphenol (GTE; 3.1, 6.3, 12.5, 25, and 50 μg/mL) and ethanol (100 mM) addition groups, and a 12.5 μg/mL phlorotannin (ECE) and ethanol (100 mM) addition group. Symbols “a” to “e” indicate significant differences. The bars not having common letters of the alphabet indicate different significant differences (p<0.05).

FIG. 2 shows malondialdehyde (MDA) concentrations in media from cultured liver calls over time in the alcoholic hepatitis model described in Example 2. The MDA concentrations are shown as the mean±standard deviation thereof. In FIG. 2, the vertical axis indicates MDA concentration in media. The horizontal axis indicates elapsed time. A diamond indicates the MDA concentration of a control group. A square indicates the MDA concentration of an ethanol addition (100 mM) group. A triangle indicates the MDA concentration of a 12.5 μg/mL phlorotannin (ECE) and ethanol (100 mM) addition group. Double asterisks (**) indicate the presence of a significant difference (p<0.01) with the control group.

FIG. 3 shows intracellular reduced glutathione (GSH) concentrations in the alcoholic hepatitis model described in Example 3. The intracellular reduced GSH concentration is shown as the mean±standard deviation thereof. The vertical axis indicates GSH concentrations, while the horizontal axis indicates elapsed time. A diamond indicates the GSH concentration of a control group. A square indicates the GSH concentration of an ethanol addition (100 mM) group. A triangle indicates the GSH concentration of a 12.5 μg/mL phlorotannin (ECE) and ethanol (100 mM) addition group. Double asterisks (**) indicate the presence of a significant difference (p<0.01) with the control group.

FIG. 4 shows an image of a control group of Example 4. The image is obtained by immunohistochemical analysis using anti-type I collagen antibody. The more darkly stained the cytoplasm, the greater the ability of the cell to synthesis collagen.

FIG. 5 shows an image of an ethanol addition group of Example 4. The image is obtained by immunohistochemical analysis using anti-type I collagen antibody.

FIG. 6 shows an image of a phlorotannin (ECE) and ethanol addition group of Example 4. The image is obtained by immunohistochemical analysis using anti-type I collagen antibody.

FIG. 7 shows recovery rates of algae extracts in the alcoholic hepatitis model of Example 5.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The present disclosure relates to a hepatitis-related therapeutic or preventive agent including a phlorotannin. Illustrative examples of the phlorotannin include, but are not limited to, one or more of eckol, phlorofucofuroeckol A, dieckol, and/or 8,8′-bieckol. In some embodiments, one or more types of the above-mentioned phlorotannins can be used alone, or as a mixture of two or more types.

In illustrative embodiments, the phlorotannin includes one or more phlorotannin extracted from one or more brown algae. Examples of brown algae include, but are not limited to, one or more of Ecklonia cava, Ecklonia kurome, Laminaria japonica, Eisenia bicycles, Eisenia arborea, Ecklonia stolonifera, Undaria pinnatifida, Undaria peterseniana, Undaria undarioides, Hizikia fusiforme, Nemacystus decipieus, Cladosiphon okamuranus, Alaria crassifolia, Sargassum horneri and Sargassum fuluvellum.

Phlorotannin can be extracted using one or more methods known in the art and/or as described herein. These methods include, but are not limited to, extraction using an alcohol solution as the extraction solvent. In some phlorotannin extraction, methods may be performed at room temperature or up to 90° C. using 25 to 100% alcohol solutions.

In illustrative embodiments, the alcohols include one or more alcohols having 1 to 4 carbon atoms such as, but not limited to, methanol, ethanol, propanol, isopropanol, and n-butanol. In illustrative embodiments, the alcohol solution is an aqueous alcohol solution.

In an illustrative method, a brown algae powder is first obtained by washing with water, drying, and crushing brown algae. The brown algae powder is then mixed into an alcohol solution and stirred for 0.1 to 4 hours at room temperature to 90° C. to extract a phlorotannin. The resulting extract solution is then filtered and the filtrate is vacuum-concentrated to obtain a brown algae crude extract. The resulting brown algae crude extract is then crushed again to obtain a brown algae crude extract powder. This brown algae crude extract powder is then mixed into an alcohol solution having a concentration of 95% or more and stirred for 0.1 to 4 hours at room temperature to 90° C. to extract phlorotannin. The resulting extract solution is then filtered and the filtrate is vacuum-concentrated to obtain a highly pure brown algae extract.

The brown algae extract having a phlorotannin of even higher purity can be obtained by carrying out the extraction step using an aqueous alcohol solution a plurality of times. Either of the extracts obtained as described above can be used as the phlorotannin described herein. In some embodiments, the brown algae crude extract may be useful, while the highly purified brown algae extract may be useful in other embodiments. In some embodiments, phlorotannin is 90 to 99% by mass of the solid fraction of the brown algae extract. In some embodiments, the phlorotannin is approximately 90%, 91%, 92%, 93%, 94%, 95%, 96%, 97%, 98%, 99%, or 100% of the solid fraction of the brown algae extract using methods known in the art and/or described herein. In some embodiments, the phlorotannin is approximately 90% to 95%, 91% to 96%, 92% to 97%, 93% to 98%, 94% to 99%, 95% to 100%, 96% to 100%, 97% to 100%, 98% to 100%, or 99% to 100% of the solid fraction of the brown algae extract. Selection of the phlorotannin of the desired purity may be determined by the person of skill in the art using methods known in the art and/or as described herein.

The phlorotannin of the present disclosure is used as a hepatitis-related preventive or therapeutic agent.

Examples of hepatitis include viral hepatitis, alcoholic hepatitis, chronic hepatitis, epidemic hepatitis, fulminant hepatitis, infectious hepatitis, and hepatocholangitis. Examples of viral hepatitis include hepatitis caused by viral infection and examples of virus include type A, B, C, D, and E virus. Hepatitis causes hepatic fibrosis as the disease progresses, eventually progressing to cirrhosis and liver cancer. Examples of liver cancer include hepatocellular carcinoma, cholangiocellular carcinoma, intrahepatic cholangiocarcinoma, benign tumor, malignant hepatoma, primary carcinoma, metastatic carcinoma, and liver cancer caused by the hepatitis.

Hepatic fibrosis occurs due to activation of liver stellate cells, a type of hepatic sinusoidal cells. When hepatic stellate cells are activated by various pathological stimuli, they differentiate into myofibroblast-like cells and produce large amounts of extracellular matrix (e.g., collagen) resulting in the progression of hepatic fibrosis.

Since the hepatitis preventive agent or therapeutic agent comprising the phlorotannin of the present disclosure can be used to prevent or treat hepatic fibrosis, it can also be used to prevent or treat the progression of hepatic fibrosis in cirrhosis and/or liver cancer. The agent of the present disclosure can be used to prevent or treat liver cancer, as well as a liver cancer preventive agent or liver cancer therapeutic agent.

The phlorotannin can be used as an active ingredient of a medicament and/or a health food at a dose of approximately 1 to 10 mg/day/kg. In some embodiments, the dose is approximately 1 mg/kg/day, 2 mg/kg/day, 3 mg/kg/day, 4 mg/kg/day, 5 mg/kg/day, 6 mg/kg/day, 7 mg/kg/day, 8 mg/kg/day, 9 mg/kg/day, or 10 mg/kg/day. In some embodiments, the dose is approximately 1 mg/kg/day to 5 mg/kg/day, 2 mg/kg/day to 6 mg/kg/day, 3 mg/kg/day to 7 mg/kg/day, 4 mg/kg/day to 8 mg/kg/day, 5 mg/kg/day to 9 mg/kg/day, 6 mg/kg/day to 10 mg/kg/day, 7 mg/kg/day to 10 mg/kg/day, 8 mg/kg/day to mg/kg/day, 9 mg/kg/day to 10 mg/kg/day. One of skill in the art would be able to determine the appropriate dose for treatments. The phlorotannin can be administered in a single administration, or divided among several administrations. It may be provided only for a single day, or over multiple days, including time courses of one week, one month, or more.

In the present disclosure, the hepatitis preventive agent or hepatitis therapeutic agent can be administered as a medicament in combination with other known liver disease therapeutic agent and/or liver protective agent. The liver cancer preventive agent or liver cancer therapeutic agent can be administered as a medicament in combination with other known liver disease therapeutic agent and liver protective agent.

In addition, the hepatitis preventive agent or hepatitis therapeutic agent of the present disclosure can also be administered as a medicament in combination with other known drugs. Examples of the other known drugs include, but are not limited to, an antivirus agent, an antibiotic agent, an antifungal agent, other hepatitis preventing agent, other hepatitis therapeutic agent, an anti-cancer agent, an anti-obesity agent, an anti-hyperlipemia agent, an anti-diabetes agent or an anti-hypertension agent. The liver cancer preventive agent or liver cancer therapeutic agent of the present disclosure can also be administered as a medicament in combination with other known drugs. Examples of the other known drugs include an antivirus agent, an antibiotic agent, an antifungal agent, other hepatitis preventing agent, other hepatitis therapeutic agent, an anti-cancer agent, an anti-obesity agent, an anti-hyperlipemia agent, an anti-diabetes agent or an anti-hypertension agent.

As a medicament, one or more additives can be included with the phlorotannin, including, but not limited to, one or more disintegration agents, diluents, lubricants, colorants, correctives, suspending agents, surfactants, dispersants, binders and coating agents. Methods for making medicaments and formulations including one or more of these additives are known in the art.

Examples of medicament administration forms can include, but are not limited to, oral, percutaneous, and injection. Specific examples of administration form include, but are not limited to, capsules, tablets, pills, gels, syrups, slurries, suspensions, powders, granules, grains, sustained-released preparations, liquids, ointments, eye drops, creams and poultices.

Hepatitis (optionally alcoholic hepatitis), or hepatitis-related ailments, can be treated by administering one or more phlorotannin described herein in the form of a medicament to a person (patient) suffering from/affected with hepatitis. In addition, progression to cirrhosis or liver cancer can be treated by administering one or more phlorotannin described herein as a medicament or health food.

In the present disclosure, hepatitis can be prevented by administering one or more phlorotannin described herein to humans in the form of a medicament or health food/drink. In addition, the progression to cirrhosis or liver cancer can be prevented by administering one or more phlorotannin described herein in the form of a medicament or health food/beverage.

In the present disclosure, the progression to cirrhosis and liver cancer caused by hepatic fibrosis can be prevented by administering one or more phlorotannin described herein to a person suffering from/affected with hepatitis in the form of a medicament and/or food/beverage.

In some embodiments, the patient may include mammals such as, but not limited to, domesticated animals, wild animals, fish, birds, and the like. In illustrative embodiments, the animals may include, but are not limited to, dogs, cats, horses, sheep, cattle, zoo animals, ducks, geese, chickens, rabbits, etc.

Examples of foods or beverages include health foods, foods for specified health uses, nutritional supplements, foods with health claims, and functional foods. Foods or beverages can be foods or beverages labeled as preventing and improving hepatitis, preventing and improving alcoholic hepatitis, improving liver function, preventing and improving cirrhosis, and/or preventing or improving liver cancer or liver disease.

As used herein, the term “preventing” or “prevention” may include an increased period of time before onset and/or detection of new and/or additional symptoms related to one or more of the diseases or disorders (optionally alcoholic hepatitis). The term “improving” and/or “treatment” may include an amelioration and/or reduction in the type and/or the severity of one or more symptoms associated with one or more of the diseases or disorders (optionally alcoholic hepatitis).

In the present disclosure, additives can be incorporated in a food or beverage. Examples of the additives include, but are not limited to, antioxidants, fragrances, organic acids, inorganic acids, salts of organic acids, salts of inorganic acids, preservatives, flavorings, sweeteners, sour agents, extracts, spices, vitamins, colorants, emulsifiers, thickeners, moisturizers, antiseptics, pH adjusters and stabilizers.

Examples of forms of foods or beverages include liquids, pastes, slurries, gels, powders, crystals, and solids. Specific examples of forms of foods or beverages include, but are not limited to, alcoholic beverages, tea drinks, coffee drinks, soft drinks, milk drinks, confections, syrups, processed fruit products, processed vegetable products, pickled vegetables, meat products, fish products, appetizers, canned and bottled goods, instant foods and beverages, tonic drinks, liver oil drops, breath fresheners and jellies.

In the present disclosure, an example of a method for ingesting the hepatitis preventive agent or hepatitis therapeutic agent of the present disclosure includes simultaneously ingesting the hepatitis preventive agent or hepatitis therapeutic agent comprising phlorotannin of the present disclosure when consuming an alcoholic beverage. In addition, consuming the alcoholic beverage comprising the hepatitis preventive agent or hepatitis therapeutic agent comprising phlorotannin makes it possible to ingest the hepatitis preventive agent or hepatitis therapeutic agent comprising phlorotannin simultaneous to consuming the alcoholic beverage.

Alcoholic beverages include, but are not limited to, beer, wine (e.g., from grapes, berries, fruits, rice, etc), hard liquor (e.g., scotch, whiskey, rum, vodka, tequila, etc.), mixed drinks, punch drinks, and the like.

There are no particular limitations on the amount of phlorotannin contained in a food or beverage of the present disclosure.

The content of phlorotannin in a food or beverage can be adjusted corresponding to the expected hepatitis preventive effects in capsules, tablets or cookies and the like so that the daily intake thereof is, for example, approximately 60 to 500 mg.

In the case of a liquid food or beverage, the content in the food or beverage is typically approximately 100 g/200 mL or less from the viewpoint of inhibiting the occurrence of irritation in the mouth and from the viewpoint of inhibiting the odor of algae in particular. In some embodiments, the content is approximately 500 g/200 mL, 400 g/200 mL, 300 g/200 mL, 200 g/200 mL, 100 g/200 mL, 80 g/200 mL, 50 g/200 mL, or 20 g/200 mL. In some embodiments, the content is approximately 20 g/200 mL to 500 g/200 mL, 20 g/200 mL to 400 g/200 mL, 20 g/200 mL to 300 g/200 mL, 20 g/200 mL to 200 g/200 mL, 20 g/200 mL to 100 g/200 mL, 20 g/200 mL to 80 g/200 mL, or 20 g/200 mL to 50 g/200 mL. In some embodiments, the content is approximately 300 g/200 mL to 500 g/200 mL, 200 g/200 mL to 400 g/200 mL, 100 g/200 mL to 300 g/200 mL, 80 g/200 mL to 200 g/200 mL, or 50 g/200 mL to 100 g/200 mL.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### EXAMPLES

The present technology is further illustrated by the following examples, which should not be construed as limiting in any way.

### Production Example 1

### Preparation of Ecklonia Cava Extract

Ecklonia cava (0.2 kg) were washed with tap water and dried. The Ecklonia cava were then crushed with a mixer (MFC SI Mill, Janke and Kunkel Ika-Wreck, Staufen, Germany) to obtain a dry powder of Ecklonia cava. The dry powder (0.1 kg) was mixed into 0.8 L of 30% aqueous ethanol solution, and stirred for 2 hours at 80° C. to extract the phlorotannin. The resulting extract solution was filtered. The filtrate was vacuum-concentrated using an evaporator to obtain 0.05 kg of Ecklonia cava crude extract. The resulting Ecklonia cava crude extract was again crushed using a mixer and then passed through a sieve to obtain an 80-mesh powder of Ecklonia cava crude extract.

The Ecklonia cava crude extract powder (0.1 kg) was mixed into 0.8 L of a 95% aqueous ethanol solution and stirred for 2 hours at 80° C. to extract the phlorotannin. The resulting extract solution was then filtered. The filtrate was vacuum-concentrated using an evaporator to obtain 0.005 kg of Ecklonia cava extract (ECE) containing 95% by mass of phlorotannin. The content of phlorotannin may be determined by, for example, the Folin-Ciocalteu method (Waterman, P. G., Mole, S. (Eds.), 1994, Analysis of Phenolic Plant Metabolites, Blackwell Scientific, Oxford.)

### Example 1

### In Vitro Alcoholic Hepatitis Model

10-week-old male Wistar rats (Japan SLC) were anesthetized with diethyl ester using the collagenase perfusion method of Moldeus, et al. (Moldeus P. et al., Isolation and use of liver cells, Methods Enzymol, 52:60-71 (1978).) Liver cells were isolated from the anesthetized rats, and were stained with Trypan blue. More than 90% of the isolated liver cells were confirmed to be viable.

The isolated liver cells were disseminated in a 35 mm plastic dish at a density of 2.5×10⁵ cells/mL in 2 mL of William's E medium containing 10% FBS. The cells were cultured overnight in a carbon dioxide gas incubator in a humidified environment at 37° C., 5% CO₂ and 95% humidity. The medium was replaced with fresh FBS-free William's E medium, and the measurements were carried out as below.

Measurement of Liver Cell Viability. Liver cell viability was measured in an Ethanol Loading test using the Neutral red method. Neutral red (a red pigment) accumulates in the endoplasmic reticulum of live cells.

The phlorotannin (ECE) obtained in Production Example 1 and 100 mM ethanol were simultaneously added to the medium containing cultured liver cells at 2.5×10⁵ cells/mL. The cells were cultured for 24 hours, and samples were taken for analysis at various time periods including 4 hours, 8 hours, 12 hours, and 24 hours.

At each time point, the medium was removed from the cultured cells, and 1 mL of 0.005% Neutral red solution was added. The 0.005% of solution was obtained by diluting 0.4% Neutral Red storage solution 80-fold with phosphate-buffered saline (PBS). The liver cells were then allowed to stand undisturbed for 2 hours in an incubator at 37° C. with 5% CO₂ atmosphere.

After removing the Neutral Red, the liver cells were washed once with an aqueous solution of 1% formaldehyde and 1% calcium chloride within 2.5 minutes. 1 mL of a 1% acetic acid-50% ethanol aqueous solution was added. The liver cells were then allowed to stand undisturbed for 30 minutes at room temperature.

The supernatant was diluted 3-fold with an aqueous solution containing 1% acetic acid and 50% ethanol. Absorbance at 540 nm was measured with a spectrophotometer (V-530, JASCO). This measurement was carried out five times to determine the average value. After having observed a difference between levels by a one-way layout analysis of variance, a multiple comparison test was carried out using the method of Tukey (National Institute of Standard and Technology (NIST) Engineering Statistics Handbook, Chapter 7.4.7.1).

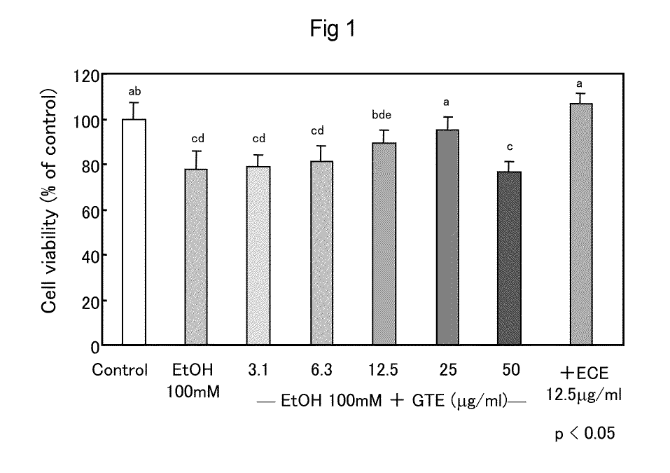

The cell viability of the liver cells was measured based on a value of 100 for a control to which neither ethanol nor phlorotannin were added. The measurement results are shown in FIG. 1. The cell viability of liver cells in the presence of green tea polyphenol (GTE, catechin, SIGMA) for also determined for comparison.

### Example 2

### Measurement of Malondialdehyde (MDA) Production

Malondialdehyde (MDA) is a secondary product of peroxides of highly unsaturated fatty acids. MDA production was measured using TBARS (Thiobarbituric Acid Reactive Substances) assay (Yagi, K., Simple assay for the level of total lipid peroxides in serum or plasma, Methods in Molecular Biology, 108:101-106 (1998).

Liver cells obtained according to the method of Example 1 were added to FBS-free Hanks medium to a density of 2.5×10⁵ cells/mL. In appropriate samples, one or the other or both of the phlorotannin (ECE) obtained in Production Example 1 and ethanol (EtOH) were added to obtain final concentrations of 12.5 μg/mL and 100 mM, respectively. The liver cells were cultured for 0 to 24 hours. Samples were collected and tested for MDA production after 4, 8, 12, and 24 hours from the start of culture.

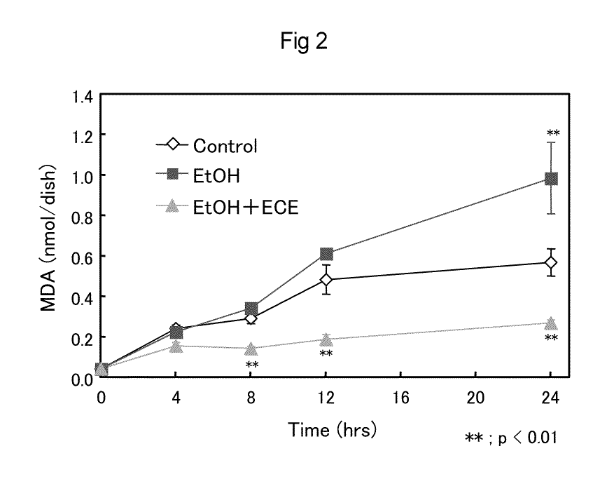

To measure MDA production, cell culture media and 500 μL of 1,1,3,3-Tetramethoxypropane standard solution were placed into a screw-top test tube. 1 mL of TBA (Thiobarbituric Acid) reagent and 15 μL of 50 mM BHT (butyl hydroxytoluene) were then added followed by mixing with a vortex stirrer. The mixture was heated in hot water for 15 minutes and then cooled over ice. The mixture was then centrifuged (3000 rpm, 10 minutes), 150 μL of the resulting supernatant were placed in a 96-well plate. Absorbance was then measured with a multi-label counter (Wallac 1420 ARVOsx) at an excitation wavelength of 485 nm and fluorescence wavelength of 535 nm. This measurement was carried out five times to determine the average value, and expressed as nmol/dish. After having observed a difference between levels by a one-way layout analysis of variance, a multiple comparison test was carried out using the method of Tukey. The measurement results are shown in FIG. 2.

### Example 3

### Measurement of Intracellular Reduced Glutathione (GSH) Levels

Glutathione (GSH) reacts with o-phthalaldehyde (OPA) to become an isoindole derivative. GSH was quantified following separation as the isoindole derivative by HPLC. Intracellular reduced (GSH) levels were measured and quantified with a spectrophotometer at an excitation wavelength of 230 nm and fluorescence wavelength of 445 nm.

Phlorotannin (ECE) obtained in Production Example 1 (12.5 μg/mL) and 100 mL of ethanol were simultaneously added to the media described in Example 1 containing 2.5×10⁵ cells/mL of cultured liver cells. The cells were then cultured for 0 to 24 hours. Samples were taken 4, 8, 12, and 24 hours after the start of culture (hour 0), and used to measure intracellular reduced GSH level. The cells were scraped off the culture dish using a 25 mM Tris-HCL solution. The cell membranes were disrupted with an ultrasound treatment followed by centrifugation. The supernatant was reacted with OPA (o-phthalaldehyde) and measured by HPLC.

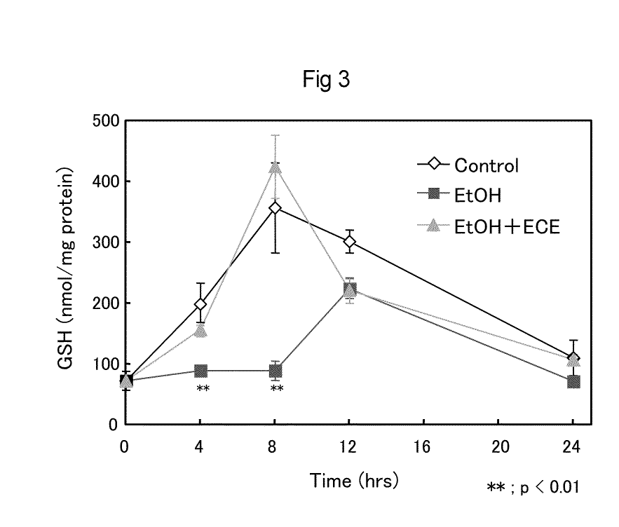

GSH was quantified by eluting and separating over a gradient created between a mobile phase A (30 mM sodium acetate, pH 6.0) and a mobile phase B (methanol: acetonitrile=92.3:7.7%) using a silica gel-based reverse phase column (Mightysil, Kanto Chemical, 15 cm, diameter: 4.6 mm). The measurements (expressed as nmol/mg protein) over time are shown in FIG. 3.

### Example 4

### Immunohistochemical Analysis Using Anti-Type I Collagen Antibody

After perfusing the livers of 13 to 14-week old male Wistar rats (Japan SLC) with pronase (Merck) and collagenase (Wako Pure Chemical Industries) solutions, liver stellate cells were obtained by density gradient centrifugation using Nycondenz solution. The liver stellate cells were suspended in 1.5 mL of DMEM (Dalbecco Modified Eagle Medium) with 10% FBS so that the cell number is 5.0×10⁵ cells/mL. The cells were cultured in a 35 mm plastic Petri dish for 2 days. Following that, the medium was replaced with FBS-free DMEM and cultured for an additional 24 hours to align the cell cycle. Next, cells were cultured in the absence of additional treatments (as control), or in the presence of 100 mM aqueous ethanol solution alone, or together with phlorotannin (ECE) from Production Example 1 (12.5 μg/mL). The cells were cultured for 24 hours, at which time, the cells were fixed using a 4% aqueous paraformaldehyde solution at 4° C. . The cells were washed three times for 5 minutes each with 0.1% Triton-X PBS solution. After blocking intrinsic peroxidase for 5 minutes using 3% aqueous hydrogen peroxide, the cells were reacted with primary antibody (anti-rat type I collagen antibody, Chemicon) for 60 minutes. After washing the cells three times for 5 minutes each using PBS, the cells were reacted with secondary antibody (biotinated anti-goat rat immunoglobulin antibody, DAKO A/S) for 30 minutes. After again washing the cells three times for 5 minutes each using PBS, the cells were reacted with an enzyme solution (horseradish peroxidase-labeled streptoavidin-biotin complex solution) for 30 minutes. After again washing the cells three times for 5 minutes each using PBS, the cells were reacted for 5 minutes using DAB solution by a peroxidase color development reaction. The cells were again washed three times for 5 minutes each using PBS. The cells were then mounted in a water-soluble mounting medium (Aquatex, Merck) to prepare specimens.

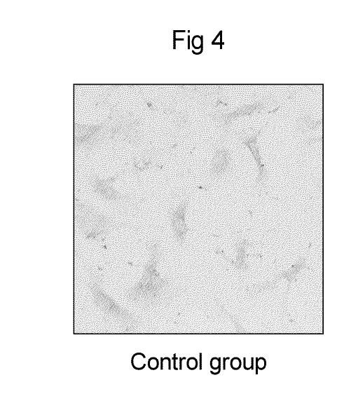

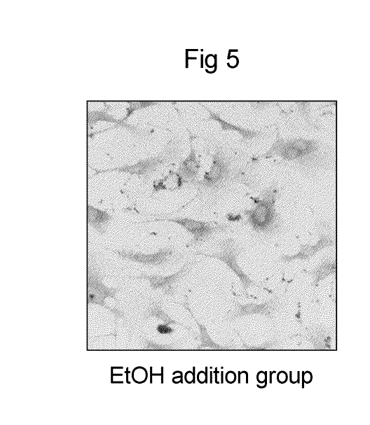

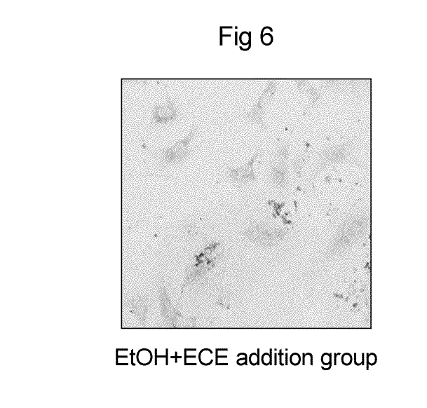

Images of the cells were obtained from a CCD digital camera (FUJIX HC-2500) connected to an inverted microscope (Olympus IX70) using PC software (FUJIX Photograph-2500 for Macintosh). The measurement results are shown in FIGS. 4 to 6. These figures show that the phlorotannin (ECE) of the present disclosure significantly inhibits an ethanol-induced increase in collagen synthesis in liver stellate cells.

### Example 5

### Comparisons of Liver Cell Viability

The Ecklonia cava crude extract obtained in Production Example 1 was dissolved in pure water to a concentration of 10 mg/mL by heating for 30 minutes at 80° C. The 10 mg/mL aqueous solution of Ecklonia cava crude extract was diluted 20-fold with pure water, and the diluted solution was added to 2 mL of William's E medium. The final concentration of the extract was adjusted to 2.5 μg/mL.

Liver cell viability was measured according to the method of Example 1. As comparative examples, Eisenia bicyclis, Hizikia fusiforme, and Laminaria japonica were extracted in a similar manner as Production Example 1 to prepare crude extracts of each. Liver cell viability was then measured for the resulting crude extracts in a similar manner as Ecklonia cava crude extract. Recovery rates were determined based on the calculation formula indicated below.

Recovery rate=[(cell viability during addition of extract and ethanol)−(cell viability during addition of ethanol only)]/[(cell viability of control)−(cell viability during addition of ethanol only)]×100

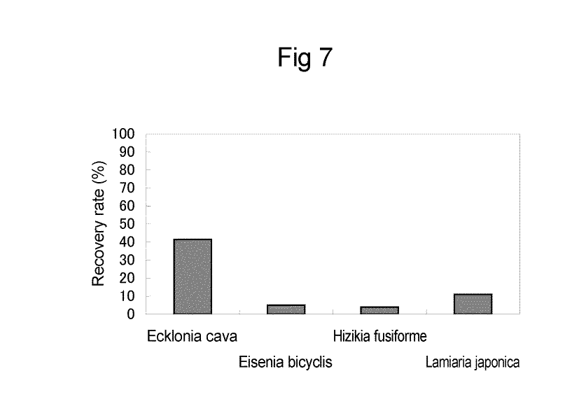

The measurement results are shown in Table 1 and FIG. 7.

TABLE 1
Recovery Rate

| | Recovery Rate |
|---|---|
| Ecklonia cava | 41% |
| Eisenia bicyclis | 5% |
| Hizikia fusiforme | 4% |
| Laminaria japonica | 11% |

### Equivalents

While certain embodiments have been illustrated and described, it should be understood that changes and modifications can be made therein in accordance with ordinary skill in the art without departing from the technology in its broader aspects as defined in the following claims.

The present disclosure is not to be limited in terms of the particular embodiments described in this application. Many modifications and variations can be made without departing from its spirit and scope, as will be apparent to those skilled in the art. Functionally equivalent methods and treatments within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A hepatitis preventive agent or hepatitis therapeutic agent comprising a phlorotannin.

2. The hepatitis preventive agent or hepatitis therapeutic agent according to claim 1, wherein the phlorotannin is a phlorotannin extracted from brown algae.

3. The hepatitis preventive agent or hepatitis therapeutic agent according to claim 1, wherein the phlorotannin is a phlorotannin extracted from Ecklonia cava.

4. The hepatitis preventive agent or hepatitis therapeutic agent according to claim 1, wherein the hepatitis is alcoholic hepatitis.

5. The hepatitis preventive agent or hepatitis therapeutic agent according to claim 1, wherein the hepatitis is cirrhosis or hepatic fibrosis.

6. A medicament comprising the hepatitis preventive agent or hepatitis therapeutic agent according to claim 1.

7. A food or beverage comprising the hepatitis preventive agent or hepatitis therapeutic agent according to claim 1.

8. An alcoholic beverage comprising the hepatitis preventive agent or hepatitis therapeutic agent according to claim 1.

9. A method for preventing or treating hepatitis comprising administering a phlorotannin in an amount effective for the prevention or treatment of hepatitis.

10. The method for preventing or treating hepatitis according to claim 9, wherein the phlorotannin is a phlorotannin extracted from brown algae.

11. The method for preventing or treating hepatitis according to claim 9, wherein the phlorotannin is a phlorotannin extracted from Ecklonia cava.

12. The method for preventing or treating hepatitis according to claim 9, wherein the hepatitis is alcoholic hepatitis.

13. The method for preventing or treating hepatitis according to claim 9, wherein the hepatitis is cirrhosis or hepatic fibrosis.

14. A liver cancer preventive agent or liver cancer therapeutic agent comprising a phlorotannin.

15. The liver cancer preventive agent or liver cancer therapeutic agent according to claim 14, wherein the phlorotannin is a phlorotannin extracted from brown algae.

16. The liver cancer preventive agent or liver cancer therapeutic agent according to claim 14, wherein the phlorotannin is a phlorotannin extracted from Ecklonia cava.

17. A medicament comprising the liver cancer preventive agent or liver cancer therapeutic agent according to claim 14.

18. A food or beverage comprising the liver cancer preventive agent or liver cancer therapeutic agent according to claim 14.

19. An alcoholic beverage comprising the liver cancer preventive agent or liver cancer therapeutic agent according to claim 14.

20. A method for preventing or treating liver cancer comprising administering a phlorotannin extracted from brown algae, wherein the phlorotannin is a phlorotannin extracted from Ecklonia cava.
